(19) United States
(12) Patent Application Publication
(10) Pub. No.: US 2024/0093267 A1
(43) Pub. Date: Mar. 21, 2024

(54) HIGH-THROUGHPUT AUTOMATED PREPROCESSING METHOD AND SYSTEM FOR NUCLEIC ACID DETECTION

(71) Applicant: Delta Electronics, Inc., Taoyuan City (TW)

(72) Inventors: Wei-Te Hsieh, Taoyuan City (TW); Chia-Yen Lin, Taoyuan City (TW); Kuang-An Wang, Taoyuan City (TW); Keng-Ting Liu, Taoyuan City (TW); Shu-Hui Huang, Taoyuan City (TW)

(21) Appl. No.: 18/370,099

(22) Filed: Sep. 19, 2023

Related U.S. Application Data
(60) Provisional application No. 63/408,394, filed on Sep. 20, 2022.

(51) Int. Cl. C12Q 1/6806; G01N 35/00; G01N 35/04

(52) U.S. Cl. CPC C12Q 1/6806; G01N 35/00663; G01N 35/00732; G01N 35/04; G01N 2035/00891; G01N 2035/0403

(57) ABSTRACT

A high-throughput automated preprocessing method and a system are applied to a nucleic acid preprocessing apparatus including a control system, a sample transfer area, a nucleic acid extraction area, and a reagent setup area. The control system includes a user interface and guides a user to set up on the user interface. In the sample transfer area, the method includes steps of: a user selecting a sampling tube type, a test protocol and an extraction protocol on the user interface, and the control system performing a sample transfer task. In the nucleic acid extraction area, the method includes steps of: the control system performing a nucleic acid extraction task based on the selected extraction protocol. In the reagent setup area, the method includes steps of: the control system performing a reagent deployment task based on the selected test protocol, and the control system performing a nucleic acid transfer task. The sample transfer area, the nucleic acid extraction area, and the reagent setup area are able to simultaneously perform preprocessing of different batches of samples.

### CROSS-REFERENCE TO RELATED APPLICATION

This application claims the benefit of U.S. Provisional Application No. 63/408,394 filed on Sep. 20, 2022 and entitled “COMBINABLE NUCLEIC ACID DETECTION SYSTEM”. The entireties of the above-mentioned patent application are incorporated herein by reference for all purposes.

### FIELD OF THE INVENTION

The present disclosure relates to a high-throughput automated preprocessing method and system, and more particularly to a high-throughput automated preprocessing method and system for nucleic acid detection.

### BACKGROUND OF THE INVENTION

The growth of global population and the development of modern transportation have not only greatly improved international trade and economic development, but also intensified the spread of infectious diseases. Especially during the global pandemic of coronavirus disease 2019 (COVID-19), the widely spread virus not only affected the global economy, but also caused a lot of inconvenience to human life. The polymerase chain reaction (PCR) test has been widely used with the COVID-19 pandemic. However, the conventional PCR test requires time and labor in sample processing, nucleic acid extraction, and subsequent amplification and testing processes.

In order to early detect the disease and prevent the spread of the disease, the molecular diagnosis has been booming in recent years. The functions of different testing devices have been integrated on the same platform to achieve the goal of so-called point-of-care testing. This fully automated nucleic acid detection platform can be used to detect infectious diseases. It not only obviates the limitation that the current nucleic acid detection can only be performed in specific medical detection centers or laboratories, but also reduces possible error judgments caused by complex operating procedures and human interpretation, and thus can provide frontline medical staffs (such as nurses and physician assistants) with rapid and accurate clinical diagnosis of infectious diseases.

The automated testing apparatus provides the graphical user interface for the user to set up and instruct the system to perform a series of sample experimental preparation and sample purification procedures. When the user selects or creates a protocol, the protocol is cross-checked for compatibility with, for example, other selected protocols and hardware capabilities of the associated automation workstations. However, since the user must set up various detailed settings for each area, the setting process is complicated, and the user must understand the relevant protocol settings and requires high expertise.

Furthermore, in occasions that require high-throughput nucleic acid detection, such as central hospitals or medical laboratories, there will be high-throughput testing demands for epidemic diseases, genetic diseases, cancers, etc. However, the conventional automated sample preprocessing operations need to be completed as a whole before proceeding to the next round. If any abnormality occurs during the operations, all actions need to be stopped for troubleshooting, and the throughput is relatively low due to the requirement of completing operations as a whole.

Therefore, how to simplify the settings for automated operations and increase the processing throughput are issues that needs to be overcome.

### SUMMARY OF THE INVENTION

An object of the present disclosure is to provide a high-throughput automated preprocessing method and a system for nucleic acid detection, which accelerates the sample preprocessing process and increases the throughput by partitional controlling the operations of sample transfer, nucleic acid extraction and reagent deployment, so as to overcome the drawbacks of the prior art.

In accordance with an aspect of the present disclosure, a high-throughput automated preprocessing method is provided. The high-throughput automated preprocessing method is applied to a nucleic acid preprocessing apparatus including a control system and three cabins connected in series and communicated with or separated from each other. These three cabins serve as a sample transfer area, a nucleic acid extraction area, and a reagent setup area, respectively. The control system includes a user interface and guides a user to set up on the user interface.

In the sample transfer area, the high-throughput automated preprocessing method includes steps of: (a1) the user selecting a sampling tube type, a test protocol and an extraction protocol on the user interface; (a2) the control system guiding the user to place consumables of a sampling tube, an extraction plate, and a tip; (a3) the control system confirming configurations of the consumables, and performing a sample transfer task; and (a4) after the sample transfer task is completed, the control system conveying the extraction plate to the nucleic acid extraction area from the sample transfer area.

In the nucleic acid extraction area, the high-throughput automated preprocessing method includes steps of: (b1) after the extraction plate is conveyed to the nucleic acid extraction area, the control system performing a nucleic acid extraction task based on the selected extraction protocol; and (b2) after the nucleic acid extraction task is completed, the control system conveying the extraction plate to the reagent setup area from the nucleic acid extraction area.

In the reagent setup area, the high-throughput automated preprocessing method includes steps of: (c1) the control system guiding the user to place consumables of a test plate, a tip, a reagent plate and reagents; (c2) the control system confirming configurations of the consumables, and performing a reagent deployment task based on the selected test protocol; and (c3) after the extraction plate is conveyed to the reagent setup area, the control system performing a nucleic acid transfer task.

Particularly, the sample transfer area, the nucleic acid extraction area, and the reagent setup area are able to simultaneously perform preprocessing of different batches of samples.

In accordance with another aspect of the present disclosure, a high-throughput automated preprocessing system is provided. The high-throughput automated preprocessing system is applied to a nucleic acid preprocessing apparatus including a control system and three cabins connected in series and communicated with or separated from each other. The three cabins serve as a sample transfer area, a nucleic acid extraction area, and a reagent setup area, respectively. The control system includes a user interface and guides a user to set up on the user interface. The high-throughput automated preprocessing system is used to perform the aforementioned high-throughput automated preprocessing method.

In an embodiment, the sampling tube type includes information of a sample type, and the control system recommends the extraction protocol based on the sample type.

In an embodiment, the control system automatically sets up a corresponding tube tray layout based on the selected sampling tube type.

In an embodiment, the sample transfer task is to transfer the sample in the sampling tube to the extraction plate.

In an embodiment, the control system confirms the configurations of the consumables through image recognition with a camera module to check whether sufficient and correct consumables are placed.

In an embodiment, in the sample transfer area, the high-throughput automated preprocessing method further includes a step of scanning a tag on the sampling tube to confirm whether the sample is correct.

In an embodiment, the nucleic acid transfer task is to transfer nucleic acids in the extraction plate to the test plate.

In an embodiment, in the reagent setup area, the high-throughput automated preprocessing method further includes a step of sealing the test plate.

In an embodiment, the control system provides a task monitoring function through the user interface, allowing the user to instantly monitor execution progresses in the sample transfer area, the nucleic acid extraction area, and the reagent setup area are.

In an embodiment, the control system displays an abnormality notification on the user interface and guides the user to troubleshoot the abnormality in the respective area where the abnormality occurs.

The above contents of the present disclosure will become more readily apparent to those ordinarily skilled in the art after reviewing the following detailed description and accompanying drawings, in which:

### BRIEF DESCRIPTION OF THE DRAWINGS

FIG. 1 shows a schematic view of the nucleic acid preprocessing apparatus of the present disclosure;

FIG. 2 shows a schematic architecture of the high-throughput automated preprocessing system of the present disclosure;

FIG. 3 shows an operation process of the high-throughput automated preprocessing system of the present disclosure;

FIGS. 4A to 4F show examples of test protocol settings in system settings;

FIGS. 5A to 5E show examples of extraction protocol settings in system settings;

FIG. 6 shows a flow diagram of the high-throughput automated preprocessing method of the present disclosure;

FIGS. 7 to 24 show examples of the user interface in the high-throughput automated preprocessing method of the present disclosure; and

FIG. 25 shows an example of abnormality notification on the user interface.

### DETAILED DESCRIPTION OF THE PREFERRED EMBODIMENT

The present disclosure will now be described more specifically with reference to the following embodiments. It is to be noted that the following descriptions of preferred embodiments of this disclosure are presented herein for purpose of illustration and description only. It is not intended to be exhaustive or to be limited to the precise form disclosed. The drawings of all the embodiments of the present disclosure are merely schematic and do not represent true dimensions and proportions.

The present disclosure provides a high-throughput automated preprocessing method and a system for nucleic acid detection. By combining hardware and software controls, various steps in nucleic acid preprocessing can be integrated in an automated pipeline. This provides the user with automated sample preprocessing services to simplify the complicated steps required in sample preprocessing. As a result, the settings on the automated nucleic acid preprocessing apparatus is streamlined, and the preprocessing workflow is accelerated, so as to enable the high-throughput testing.

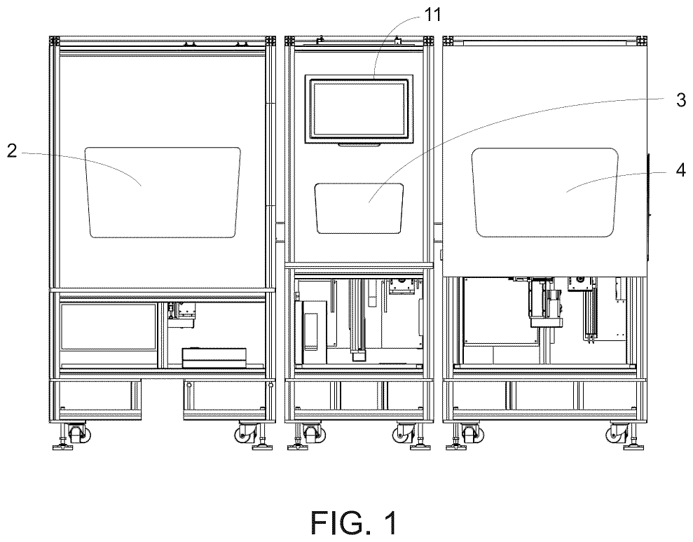

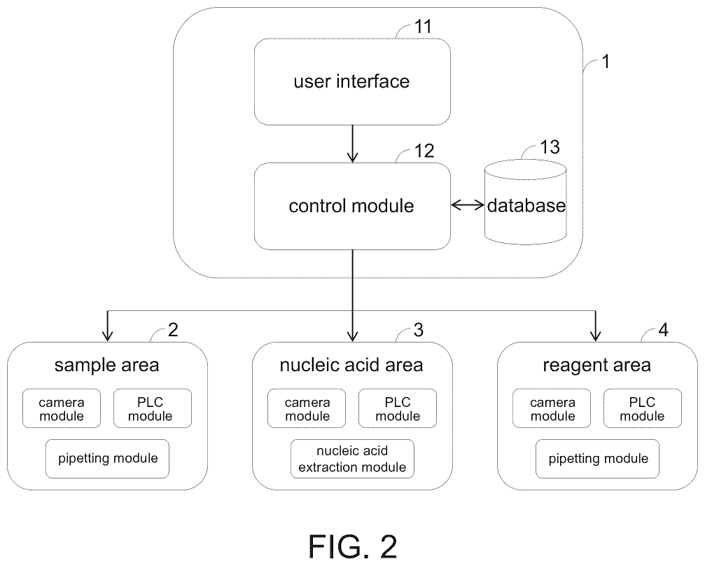

The high-throughput automated preprocessing method and system of the present disclosure are applied to a specially designed nucleic acid preprocessing apparatus, and the high-throughput automated preprocessing system is used to perform the high-throughput automated preprocessing method. FIG. 1 shows a schematic view of the nucleic acid preprocessing apparatus of the present disclosure, and FIG. 2 shows a schematic architecture of the high-throughput automated preprocessing system of the present disclosure. As shown in FIGS. 1 and 2, the nucleic acid preprocessing apparatus includes a control system 1 and three cabins that can be connected in series and communicated with each other or separated from each other. The three cabins serve as a sample transfer area (referred as sample area) 2, a nucleic acid extraction area (referred as nucleic acid area) 3, and a reagent setup area (referred as reagent area) 4, respectively. The sample area 2 is used for transferring a sample in a sampling tube to an extraction plate, the nucleic acid area 3 is used for nucleic acid extraction, and the reagent area 4 is used for preparing and mixing reagents and dispensing and distributing the reagents and the nucleic acids to a test plate. The three cabins can independently perform the task of the current cabin, and after the current cabin completes the sample processing of the current batch of samples, the processed samples are conveyed to the next cabin, and the current cabin can process the next batch of samples, thereby increasing the processing throughput. The operations of the three cabins are controlled by the control system 1. The control system 1 includes a user interface 11, a control module 12, and a database 13. The user interface 11 is a human-machine interface used to guide the user to perform settings and controls on the user interface 11, and is in charge of the communication and interaction between the user and the control module 12 for experimental preparation, monitoring and system settings. The control module 12 is in charge of the data transmission and the control process between the front-end and the back-end, and can convert the user's settings and controls into instructions for execution on the back-end. The database 13 is in charge of the storage and access of the system-related data.

The sample area 2 includes a camera module, a programmable logic controller (PLC) module, and a pipetting module. The nucleic acid area 3 includes a camera module, a PLC module, and a nucleic acid extraction module. The reagent area 4 includes a camera module, a PLC module, and a pipetting module. The camera module is used to capture images of consumables such as sampling tubes, extraction plates, test plates, tips, and reagent plates, so that the control system can check whether the correct and sufficient consumables are placed, or whether the mechanism has successfully grabbed/released the consumables. The camera module, or other barcode identification module, may scan the tag on the sampling tube to confirm whether the sample is correct. The PLC module is used to receive instructions from the control system for controlling various motor actions. The pipetting module is used to transfer the liquid samples, the nucleic acids and various reagents. The nucleic acid extraction module is used to extract the nucleic acids from the sample for downstream nucleic acid detection.

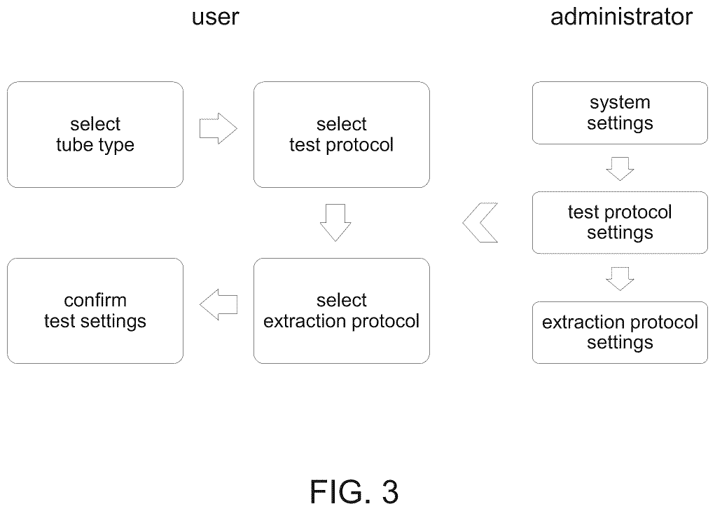

FIG. 3 shows an operation process of the high-throughput automated preprocessing system of the present disclosure. As shown in FIG. 3, the operation process can be divided into a user-end process and an administrator-end process. The administrator-end process is mainly system settings, including test protocol settings and extraction protocol settings. The test protocol settings mainly involve setting up the test recipes (such as COVID-19 testing or influenza testing) and relevant settings. For example, the setting items include setting the test name and the supported machine model, setting the volumes of the nucleic acids and the reagents, setting the test target, setting the standards, etc. The extraction protocol settings mainly involve setting up the relevant extraction parameters (such as heating temperature and mixing time), and the parameters may be adjusted based on the sample type (such as blood, respiratory swab, urine and stool) and the test target (such as bacteria or viruses, and to extract DNA or RNA). For example, the setting items include setting the test name, setting the sample type, setting the extraction parameters, etc. The user-end process is mainly experimental settings. When the system settings on the administrator-end are completed and the user-end intends to conduct experiments, the experimental settings include selecting the sampling tube type, selecting the test protocol, selecting the extraction protocol, confirming the test settings, etc.

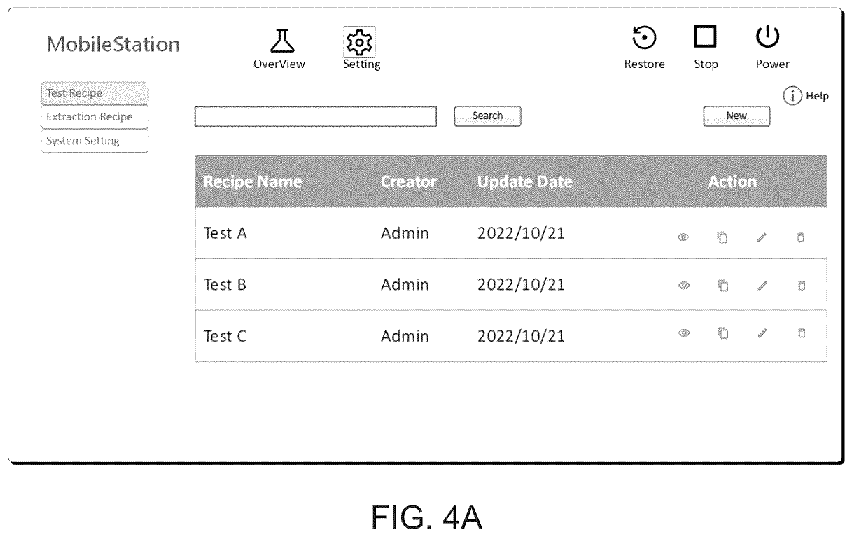

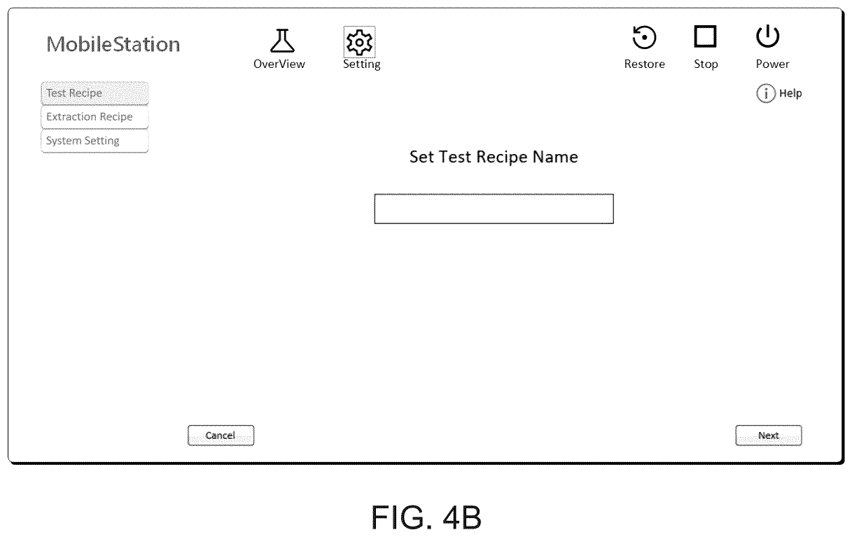

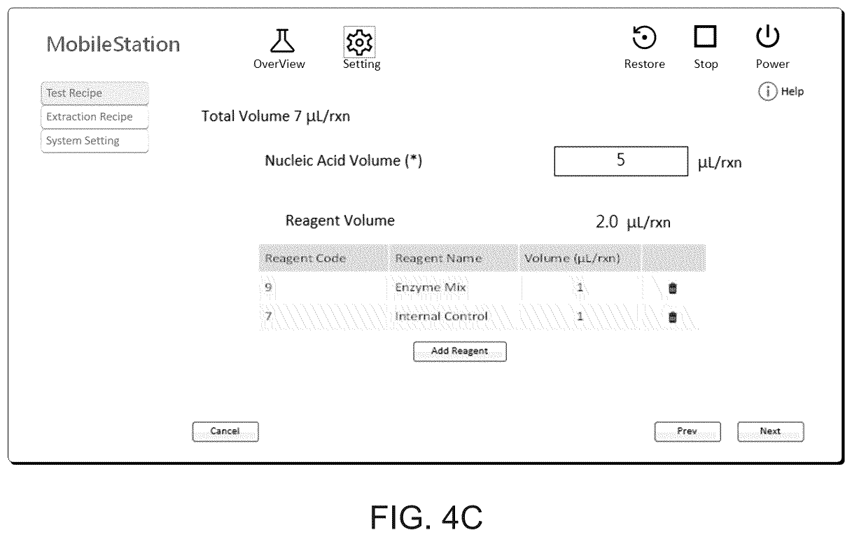

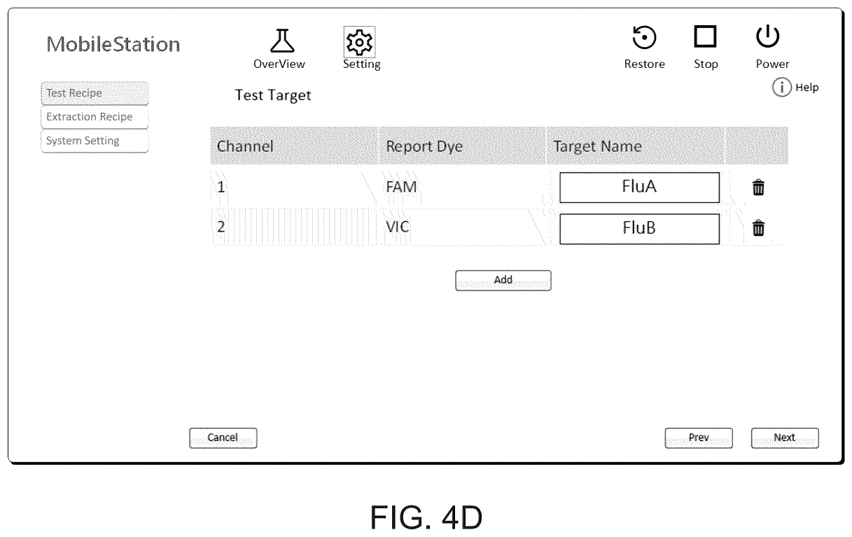

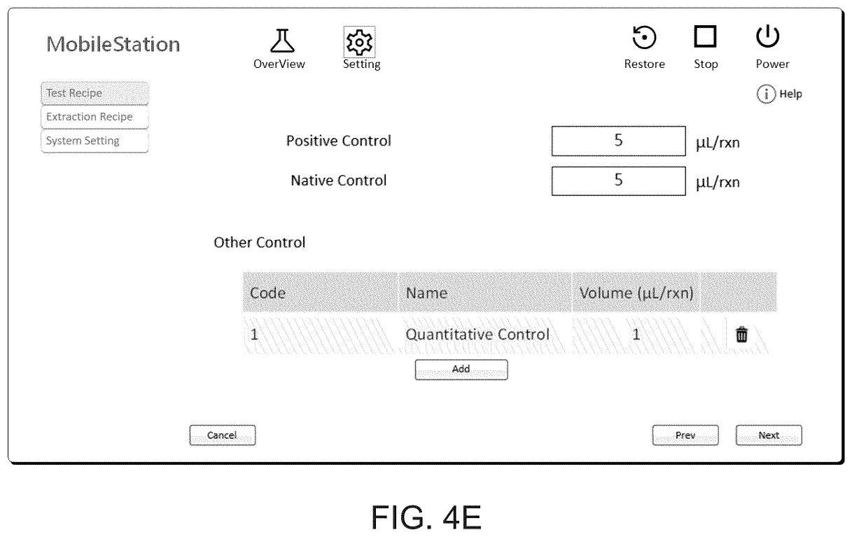

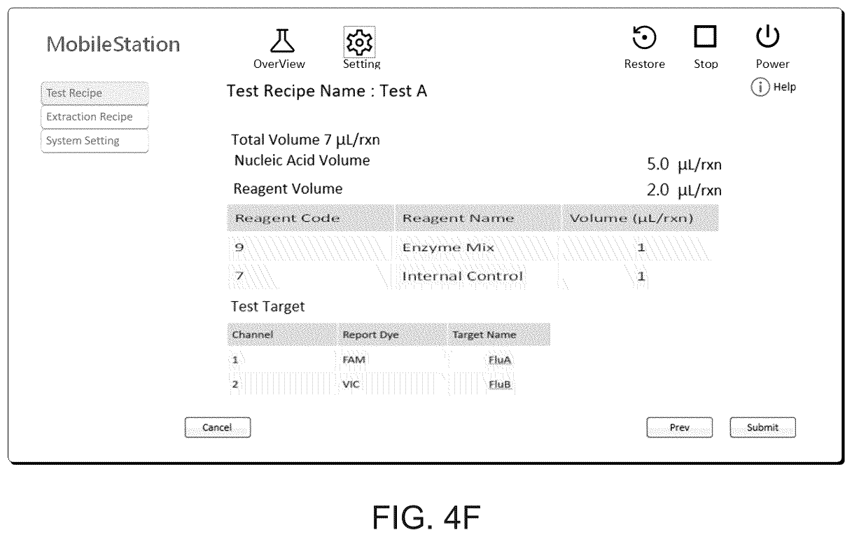

FIGS. 4A to 4F show examples of the test protocol settings in the system settings. Taking test reagent setting as an example, when the extracted nucleic acids are subject to PCR testing, the control system can preset corresponding test reagents for different test targets, or the user can add test reagents according to requirements. First, as shown in FIG. 4A, the user enters the test recipe list, and clicks the “New” button to enter the test recipe adding process, and the control system will guide the user to set up through the user interface. As shown in FIG. 4B, the test recipe name is to be input, and the supported machine model may also be select or input. Then, the reagent formula is to be set up. As shown in FIG. 4C, the nucleic acid volume is first input, then the reagents required for the formula is selected and the reagent volume is input. Then the test target is further set up after the reagent formula is set up. As shown in FIG. 4D, the reporter dye, such as various fluorescent markers FAM, VIC, ABY, JUN, Cy5, Cy5-5 with different wavelengths, is selected, and the target name is input. Subsequently, the standard setting is to be performed. As shown in FIG. 4E, a preset positive control and a preset negative control are selected or other controls may be added, and then the volumes of the controls are input. The negative control may also be any reagent in the reagent formula, and can be selected from the associated reagent option. Finally, as shown in FIG. 4F, the detailed settings of the test recipe are shown, and after the user confirms the settings, the test reagent setting is completed.

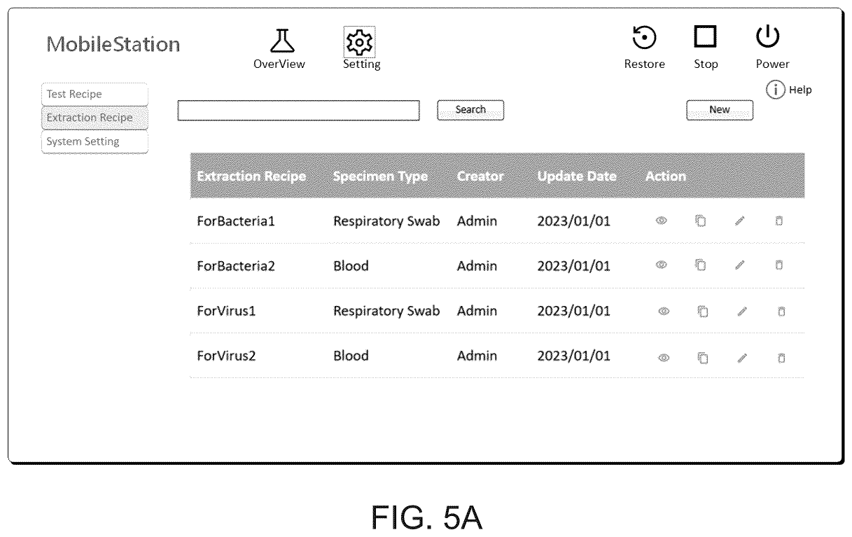

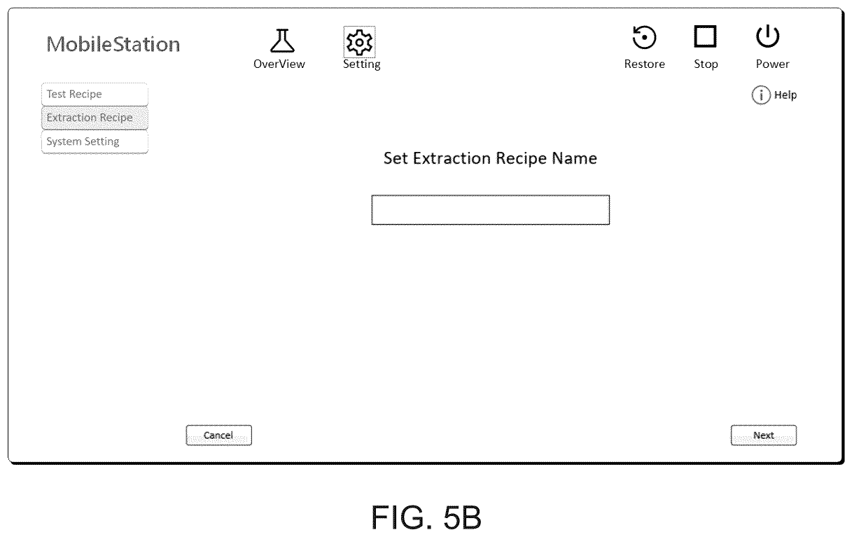

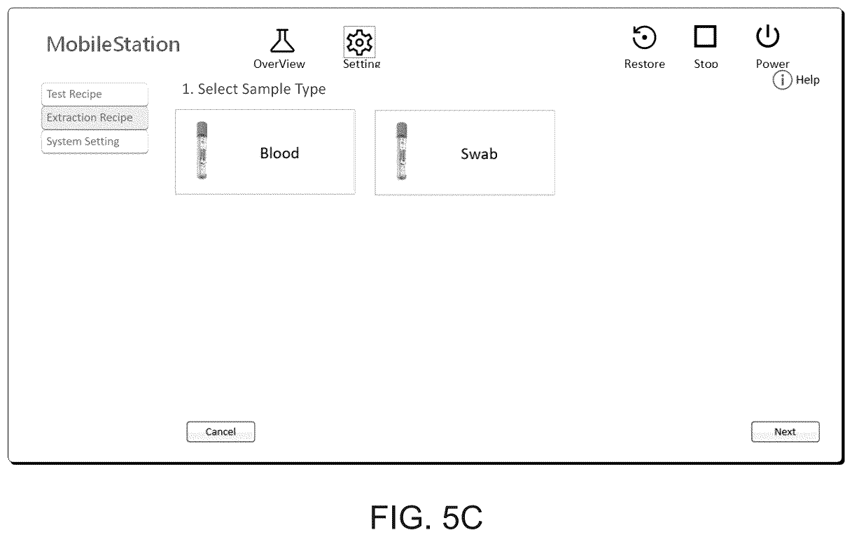

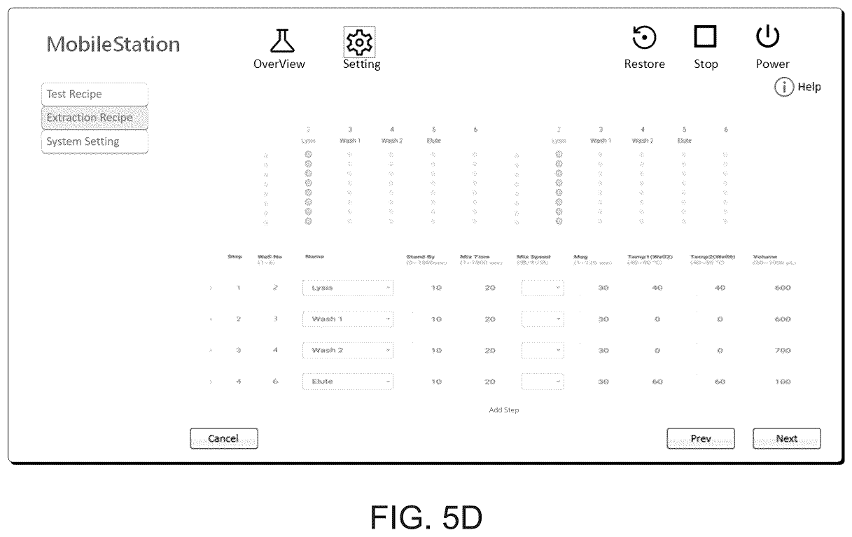

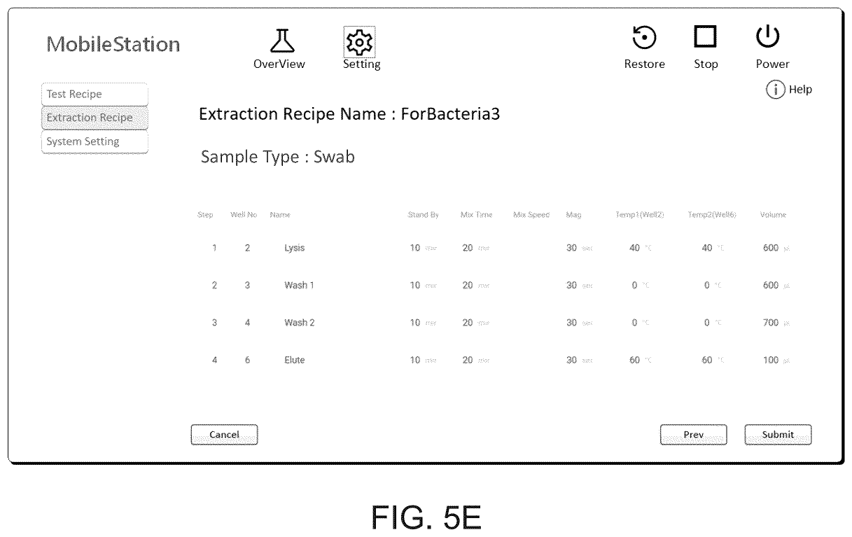

FIGS. 5A to 5E show examples of the extraction protocol settings in the system settings. Taking extraction reagent setting as an example, as shown in FIG. 5A, the user first enters the extraction recipe list, and clicks the “New” button to enter the extraction recipe adding process, and the control system will guide the user to set up through the user interface. As shown in FIG. 5B, the extraction recipe name is to be input, and as shown in FIG. 5C, the sample type, such as blood, respiratory swab, urine and stool, is selected. Then, as shown in FIG. 5D, the extraction parameters are further set up. The control system guides the user through the user interface to select/add steps, and set up well numbers, operation times, standby times, mixing times, mixing speeds, magnetic times, temperatures and volumes of the steps. For example, the extraction plate shown in FIG. 5D is a 96-well extraction plate, which can be divided into two blocks on the left and right. Each block includes 8 horizontal well groups, and each well group has 6 wells used to perform an extraction process for a sample. The first well is for accommodating a stirring rod (e.g., magnetic rod sleeve), the second well is for accommodating the sample and performing the lysis step, the third and the fourth wells are for performing the wash steps, the fifth well is for performing the elution step, and the sixth well is for accommodating the extracted nucleic acids. The control system can set up relevant extraction parameters for the second well to the fifth well. Finally, as shown in FIG. 5E, the detailed settings of the extraction recipe are shown, and after the user confirms the settings, the extraction protocol setting is completed.

In an embodiment, the control system has the function of recommending extraction protocols. When the user selects the sampling tube type (including sample type information) and the test protocol, the control system will list available extraction protocols on the user interface based on the selected sample type, and the usage counts of the extraction protocols for the selected test protocol in the past is served as a sorting basis to recommend the user. In other words, the control system will sequence the extraction protocols based on the usage counts of the extraction protocols when the same test protocol was selected by the user in the past, so the more commonly used extraction protocols for the selected test protocol will list first for the user to select.

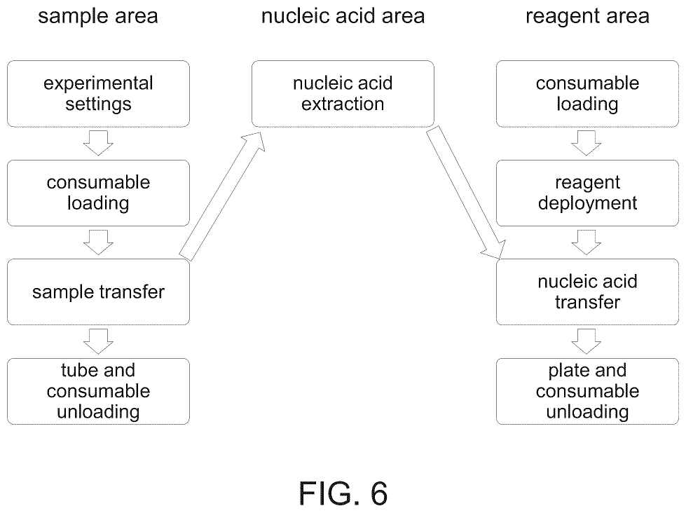

The high-throughput automated preprocessing method of the present disclosure is used with the three cabins of the nucleic acid preprocessing apparatus to control the sample preprocessing tasks in the sample area, the nucleic acid area, and the reagent area. When the user intends to start a new experimental setting, the various settings of the experimental preparation can be set up through the user interface. The various settings include selecting the sampling tube type, selecting the test protocol, selecting the extraction protocol, and confirming the experimental settings. Thereby, the automated preprocessing of the nucleic acid samples can be completed according to the guidance of the control system, and the flow diagram of the high-throughput automated preprocessing method of the present disclosure is shown in FIG. 6.

In the sample area, based on the sampling tube type selected by the user, the control system provides the corresponding tube tray layout and test plate settings, and calculates the quantity of available PCR reaction wells and corresponding required consumables. After completing the relevant settings and consumable loading, the control system will start the sample transfer task from the sampling tube to the extraction plate according to the configuration. During the sample transfer process, the control system instantly displays the tube tray number, the progress, and the remaining time for the current transferring. After the sample transfer is completed, the control system conveys the extraction plate to the nucleic acid area. Finally, the user is guided to unload the sampling tubes and the consumables.

The nucleic acid area receives the extraction plate conveyed from the sample area, and performs nucleic acid extraction task based on the extraction protocol selected in the experimental settings. During the extraction process, the control system instantly displays the extraction plate number, the extraction stage, the progress, and the remaining time for the current extraction. After the extraction is completed, the control system conveys the extraction plate to the reagent area.

The operation in the reagent area can be divided into two stages. In the first stage, when the user completes the experimental settings, the reagent area starts the tasks of consumable loading and reagent deployment. Based on the test protocol, the control system automatically displays the test plate configuration and the reagent plate configuration. The test plate configuration displays the test plate layout and the test recipe to be performed, and the reagent plate configuration displays the required reagent names and volumes calculated based on the test plate configuration. The control system deploys the required reagents based on the reagent plate configuration and dispenses the reagents to the reaction wells of the test plate. During the reagent deployment process, the control system instantly displays the progress and the remaining time of the reagent deployment in the reagent area. In the second stage, after the reagent area receives the extraction plate conveyed from the nucleic acid area, the nucleic acid transfer task is started. The control system transfers the extracted nucleic acids in the extraction plate to the test plate based on the test plate configuration, and further mixes the extracted nucleic acids with the reagents in the test plate. During the nucleic acid transfer process, the control system instantly displays the nucleic acid transfer progress and the remaining time in the reagent area. After the nucleic acid transfer is completed, the user is guided to unload the test plates and the consumables.

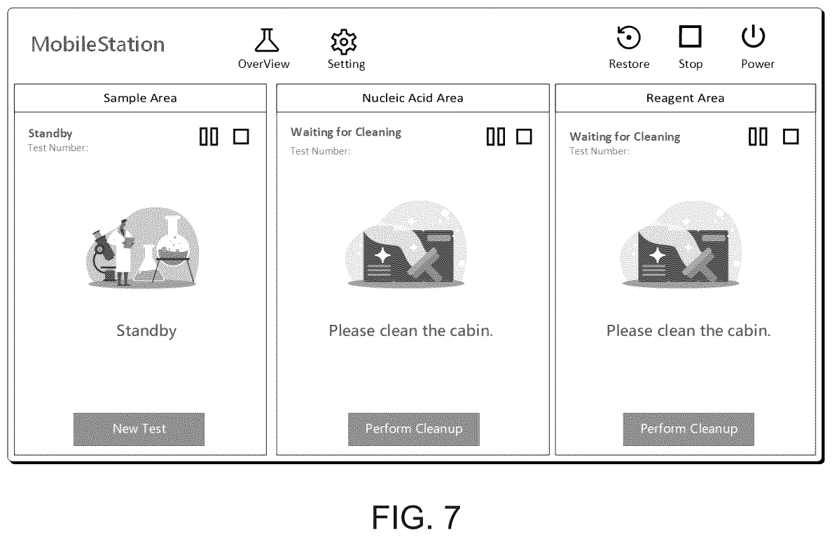

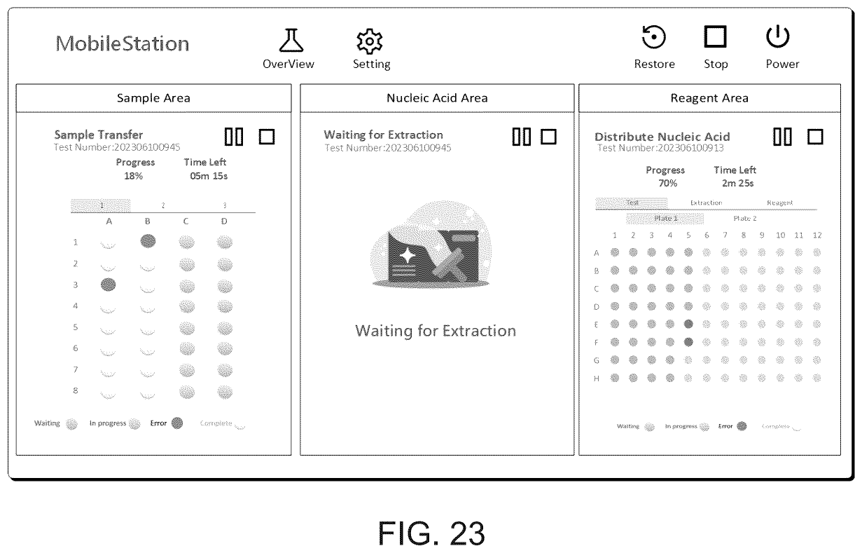

The high-throughput automated preprocessing method of the present disclosure will be demonstrated below with the user interface shown in FIGS. 7 to 24. First, as shown in FIG. 7, before a new experimental task starts, the sample area, the nucleic acid area, and the reagent area are in standby state, or the option of “Perform Cleanup” is displayed. If it has been more than a predetermined time since the last cleaning, the user can choose to perform disinfection and cleaning actions to the cabin.

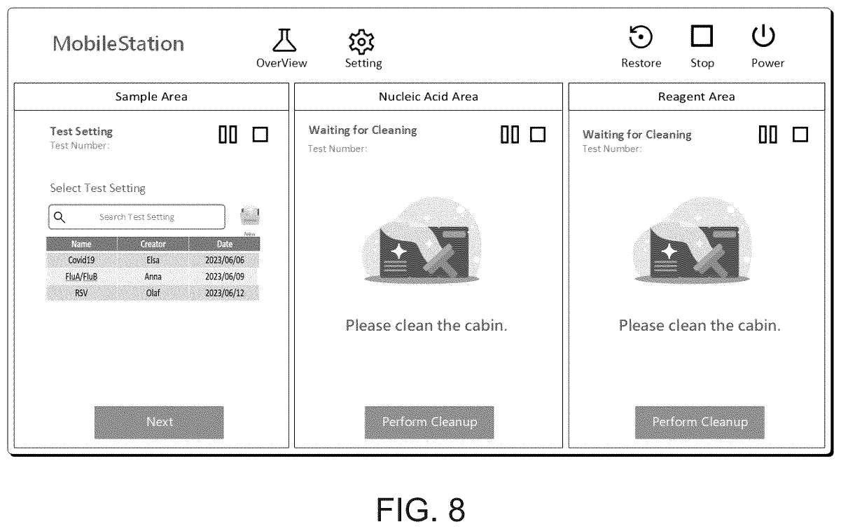

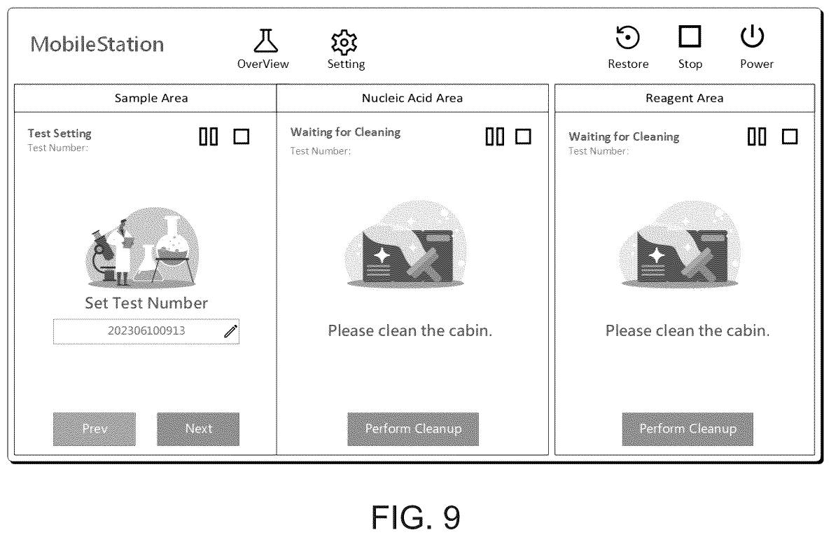

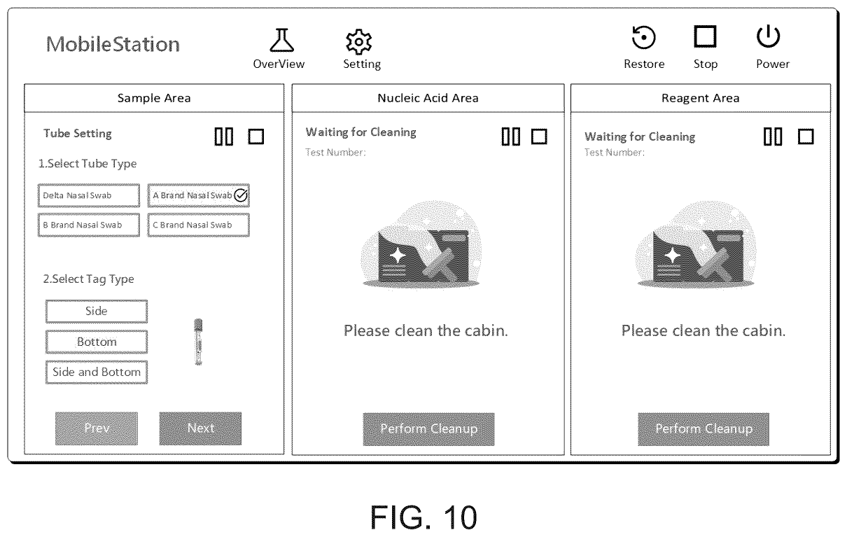

Then in the sample area, as shown in FIG. 8, the user starts a new test setting through the user interface. The user can select a prestored test setting or start a new setting. Subsequently, as shown in FIG. 9, when the user selects a prestored test setting or completes a new setting, the control system guides the user to set up the test number through the user interface. Then, as shown in FIG. 10, the control system guides the user to select the sampling tube type and the tag type. The control system has preset various brands of sampling tube types. After the user selects the sampling tube type, the location of the tag is further selected for downstream barcode scanning of the tag.

In an embodiment, the sampling tube type also includes the sample type information. When the user selects the sampling tube type, the control system also catches the sample type, which can be used for the subsequent extraction protocol setting. For example, as shown in FIG. 10, when the user selects the tube type of “Delta Nasal Swab”, the information that the sample type is nasal swab is also included. In addition, since different sampling tubes may have different sizes, when the sampling tubes are placed in the sample area, they will have different configurations, and the control system will automatically set up corresponding tube tray layouts based on the tube type selected by the user. The layout may be 8×12 tube tray layout, 6×8 tube tray layout, or 4×8 tube tray layout.

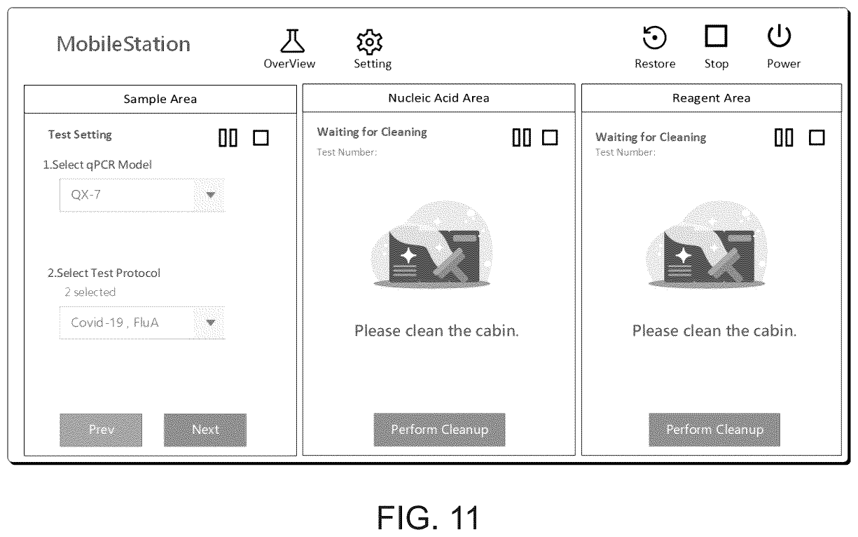

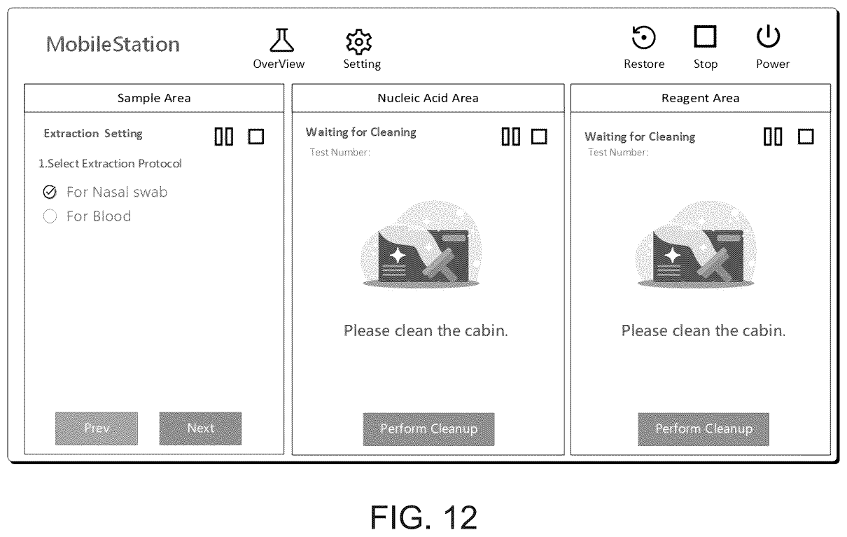

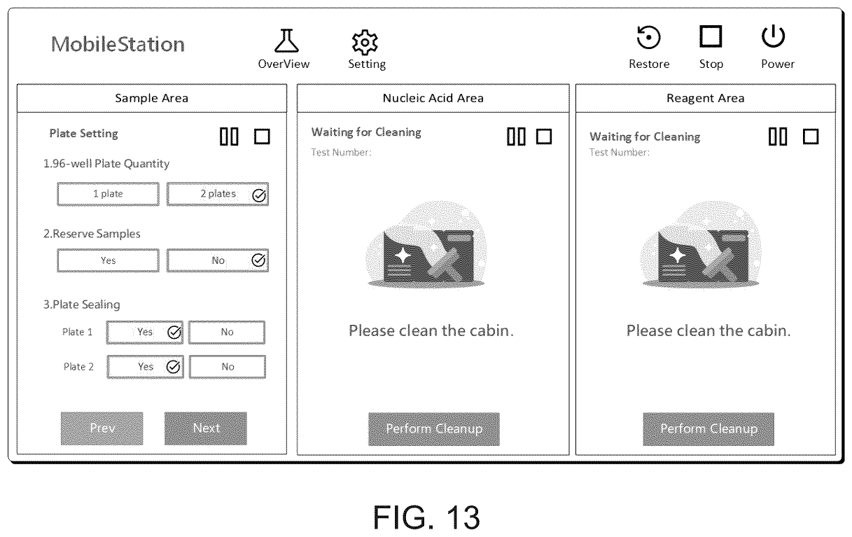

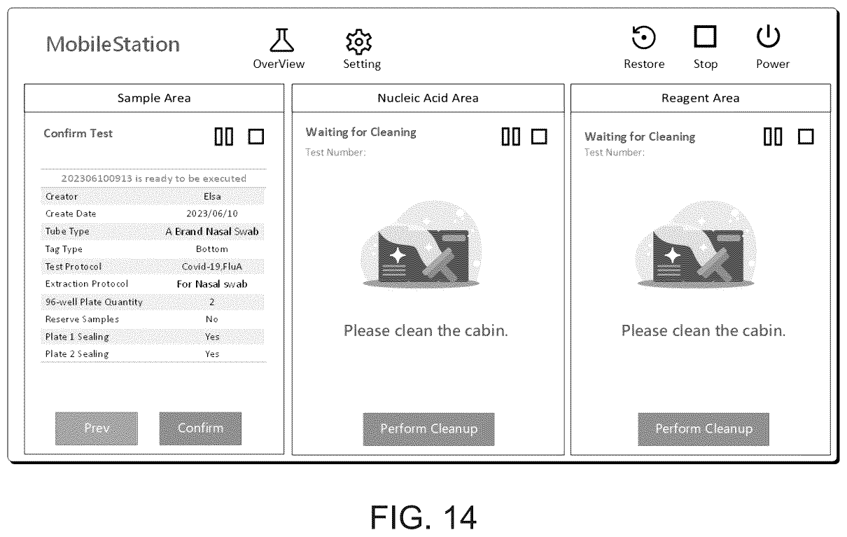

Then, as shown in FIG. 11, the control system guides the user through the user interface to select the PCR machine model and the test protocol. As shown in FIG. 12, the control system guides the user to select the extraction protocol through the user interface. Subsequently, as shown in FIG. 13, the control system guides the user to select the quantity of the test plate and the usage setting through the user interface. For example, if the quantity of 96-well plate is 2 and to reserve samples is selected, one of the two 96-well plates will be used to perform the test and the other will be used to reserve samples. While if to reserve samples is not selected, both 96-well plates will be used to perform the test. In addition, the user can also decide whether the test plate is to be sealed. Afterwards, the experimental settings are to be confirmed. As shown in FIG. 14, the control system lists various experimental settings on the user interface and guides the user to confirm the various settings of the task and then execute the task.

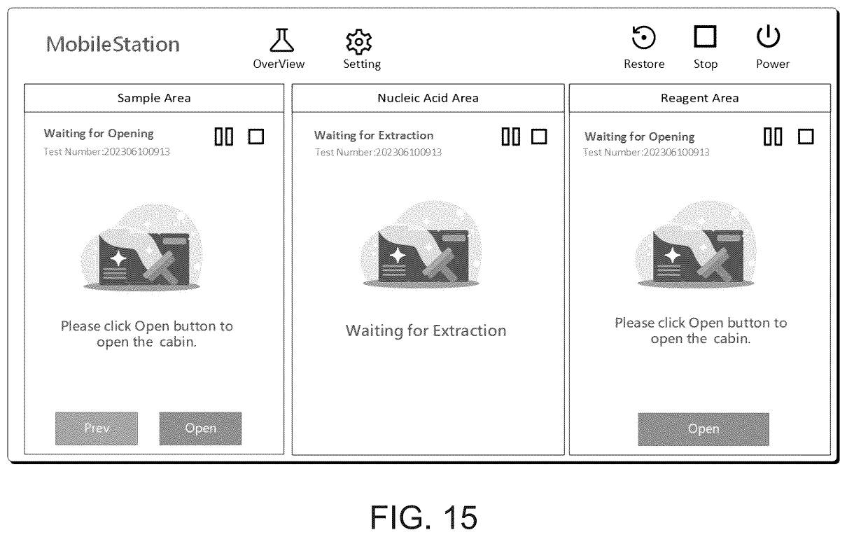

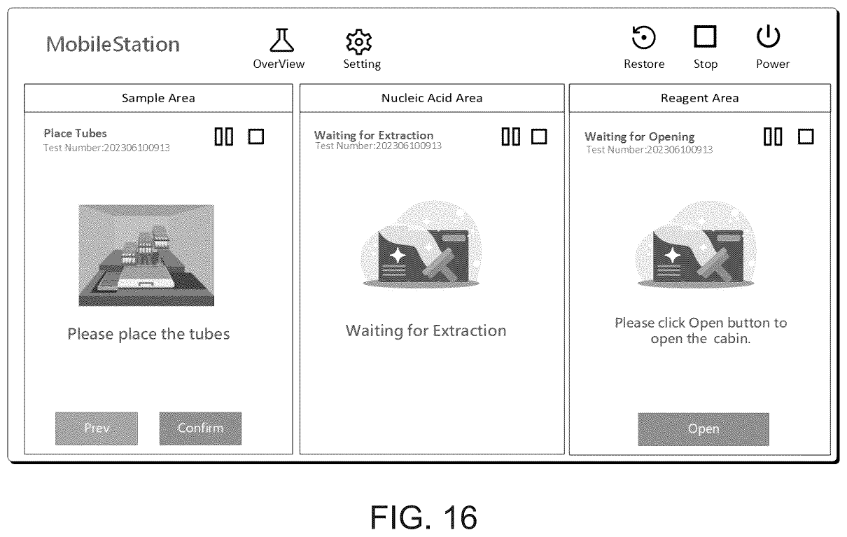

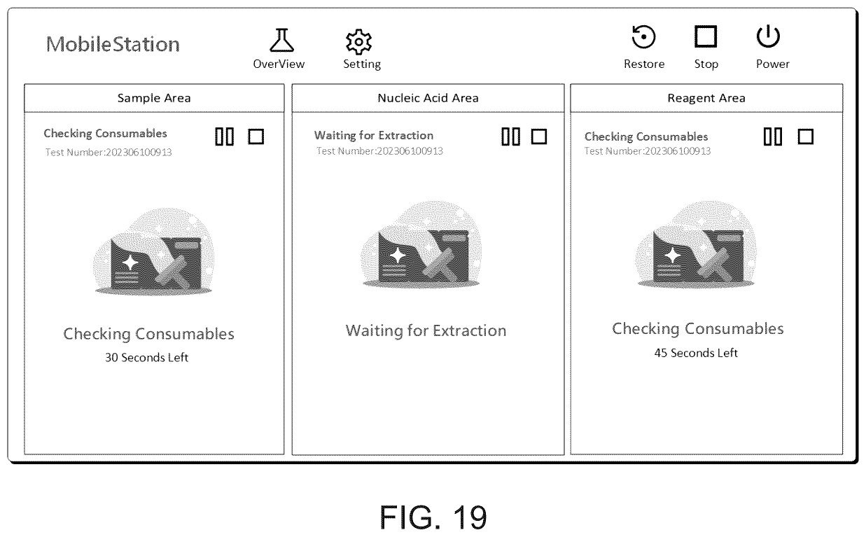

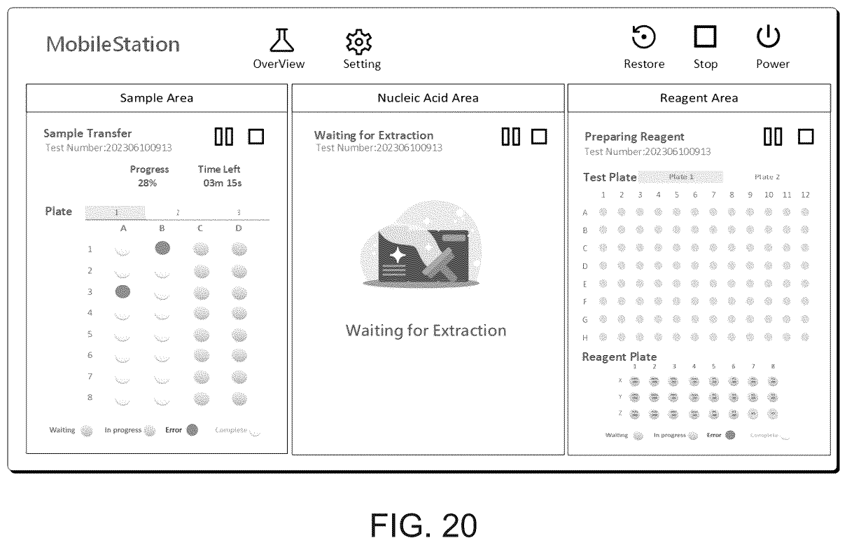

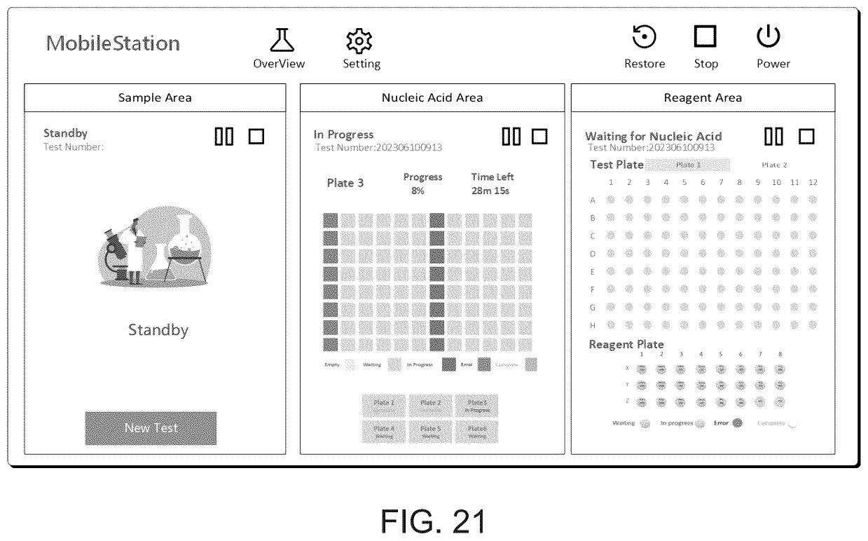

Later, as shown in FIG. 15, the control system guides the user through the user interface to open the cabin. As shown in FIGS. 16 to 18, the control system guides the user through the user interface to place various consumables such as the sampling tubes, the extraction plates, and the tips. Then, as shown in FIG. 19, after the user completes the consumable loading, the control system captures images by the camera module and checks whether sufficient and correct consumables are placed through image recognition. Subsequently, as shown in FIG. 20, after the relevant configuration is confirmed, the control system starts the sample transfer task, and instantly displays the transfer progress and the remaining time for experiment monitoring. Later, as shown in FIG. 21, after the sample transfer task is completed, the control system conveys the extraction plate to the nucleic acid area and confirms that the user has cleared the sample area, and then the sample area enters the standby state.

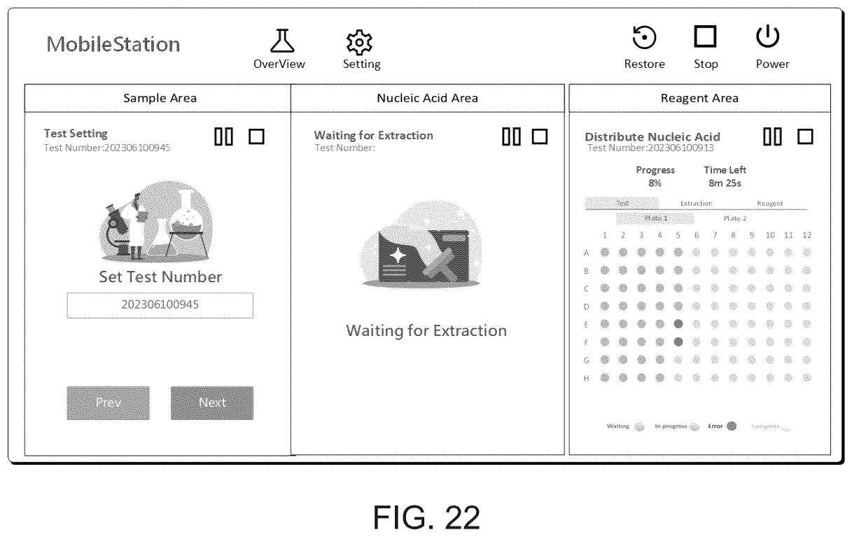

In the nucleic acid area, the following workflow is included. First, as shown in FIG. 7, the control system guides the user through the user interface to disinfect and clean the nucleic acid area. Then, as shown in FIG. 15, the nucleic acid area enters the waiting state after the disinfection and cleaning is completed. Afterwards, as shown in FIG. 21, after the sample transfer task is completed in the sample area, the nucleic acid area receives the extraction plate conveyed from the sample area and starts loading the magnetic rod sleeve for the extraction operation, and the control system instantly displays the progress and the remaining time for experiment monitoring. Later, as shown in FIG. 22, after the nucleic acid extraction task is completed, the control system conveys the extraction plate to the reagent area, and then the nucleic acid area enters the standby state to wait for the next task.

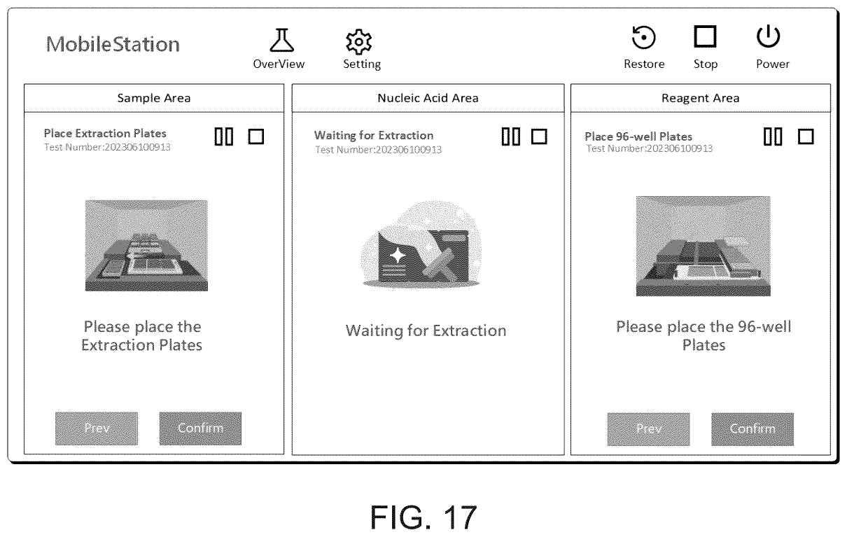

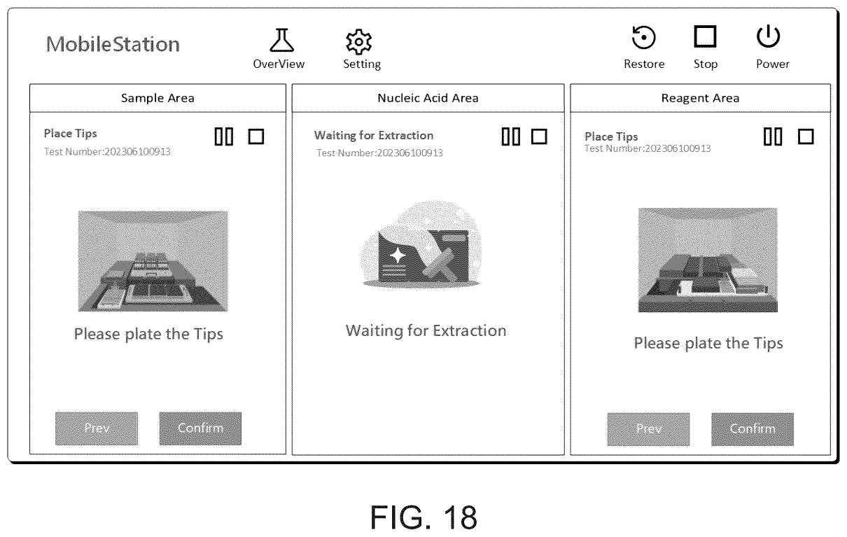

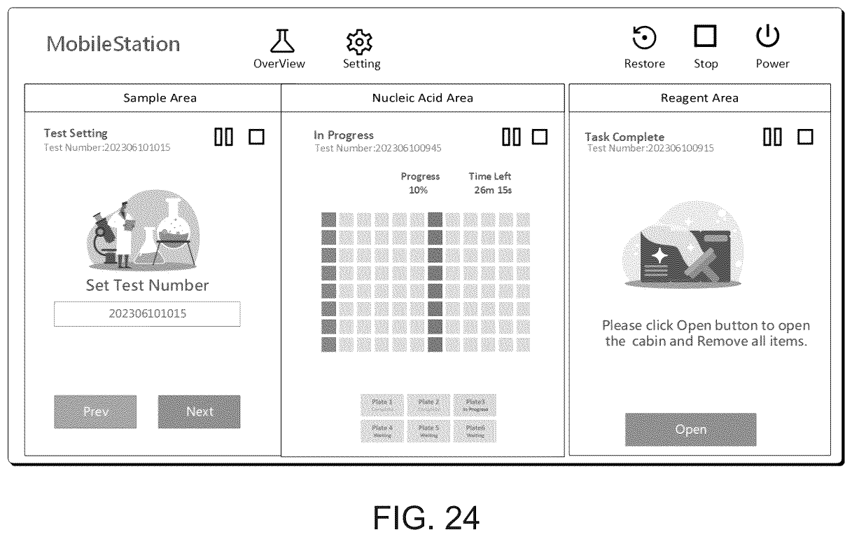

In the reagent area, the following workflow is included. First, as shown in FIG. 7, the control system guides the user through the user interface to disinfect and clean the reagent area. Then, as shown in FIG. 15, the control system guides the user through the user interface to open the cabin. As shown in FIGS. 17 and 18, the control system guides the user through the user interface to place various consumables such as the test plates, the tips, the reagent plates and the reagents. Later, as shown in FIG. 19, after the user completes the consumable loading, the control system captures images by the camera module and checks whether sufficient and correct consumables are placed through image recognition. Afterwards, as shown in FIG. 20, after the relevant configuration is confirmed, the control system starts the reagent deployment. Based on the test quantity of the test plate configuration, the control system calculates the required reagent volumes, and automatically display the reagent plate configuration on the user interface for the user to review the required reagent names and volumes. After the reagent deployment task to prepare the reagents and transfer the prepared reagents to the test plate is completed, the reagent area enters the nucleic acid waiting state, as shown in FIG. 21. Then, as shown in FIG. 22, when the extraction plate is conveyed from the nucleic acid area to the reagent area, the task of transferring the nucleic acids to the test plate starts. As shown in FIG. 24, after the nucleic acid transferring task is completed, the test plate is sealed to complete the entire preprocessing operations. Subsequently, the control system guides the user through the user interface to unload the test plates and other consumables.

From the above, sine the sample area, the nucleic acid area, and the reagent area are individually controlled, different batches of sample preprocessing operations can be performed at the same time. For example, when the reagent area is mixing the nucleic acids and the reagents of batch A, the nucleic acid area can simultaneously perform the nucleic acid extraction of batch B, and the sample area can simultaneously perform the sample transfer of batch C, so the sample preprocessing operations of three baches of samples can be simultaneously performed in a pipeline manner. Therefore, the present disclosure provides the high-throughput automated preprocessing for nucleic acid detection, which accelerates the sample preprocessing process, significantly reduces time and labor costs, and further increases the downstream detection throughput.

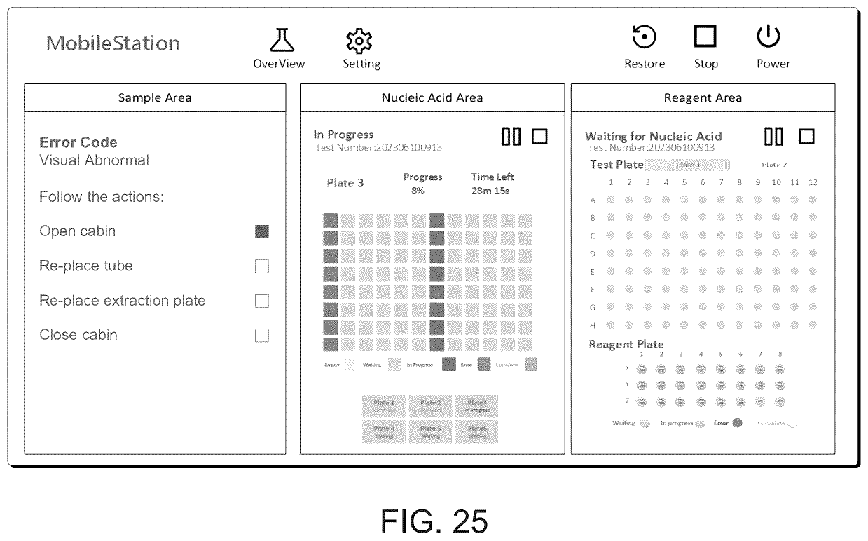

On the other hand, due to the partitioning operations of the sample area, the nucleic acid area, and the reagent area, if an abnormality occurs during the operations, the troubleshooting task can be performed separately in the abnormal area only without affecting the operations of the other areas, thereby reducing the impact of troubleshooting issue. For example, when an abnormality occurs in the sample area, the control system will display an abnormality notification on the user interface (as shown in FIG. 25) and guide the user to troubleshoot the abnormality. At the same time, the nucleic acid area and the reagent area continue to operate.

Furthermore, the present disclosure provides the user interface which is easy for the user to select and set up, thereby reducing the user learning curve. After the user selects the sampling tube type and the test protocol, the control system will automatically recommend the extraction protocol. Also, after the experimental settings are completed, the control system provides loading guidance to instruct the user of the placement of the required consumables such as the sampling tubes, the extraction plates, the test plates, the reagent plates and the tips, and uses image recognition for confirmation and further notices the user of the area with abnormal placement. During the experiment processes, the control system also provides the function of task monitoring through the user interface for the user to instantly monitor progresses and abnormality in the sample area, the nucleic acid area, and the reagent area.

In addition, the present disclosure provides the function of customizing the reagent formulas. The user can add or modify the recipe content and the standard setting of the test reagent in the system settings according to the requirements. During the experiment processes, the control system deploys the required reagents based on the quantity of the sampling tubes and the test reagent setting and dispenses the reagents to the corresponding reaction wells of the test plate. The present disclosure also supports multiple specifications of the sampling tubes, and the control system can automatically set up corresponding tube tray layout based on the tube type selected by the user. Moreover, the present disclosure further supports multiple test targets, so multiple tests can be performed on the same sample at the same time. The control system can guide the user to set up the test protocol for each sampling tube, and then automatically display the test plate configuration and the reagent plate configuration. The information of the test plate configuration can also be output for use by the downstream PCR machine.

In conclusion, the present disclosure provides a high-throughput automated preprocessing method and a system for nucleic acid detection. Due to the partitioning operations and controls of the sample area, the nucleic acid area, and the reagent area, the high-throughput automated preprocessing for nucleic acid detection is achieved, and the impact of troubleshooting issue is reduced. The present disclosure also provides the user interface which is easy for the user to select and set up. The user only needs to perform simple test-guided setting steps, including selecting the sampling tube type, selecting the test protocol, selecting the extraction protocol, and confirming the experimental settings, and follow the guidance of the user interface, and then the tasks of nucleic acid extraction and transferring the extracted nucleic acids to the test plate for downstream nucleic acid detection are easily completed. Therefore, the high-throughput automated preprocessing method and system of the present disclosure greatly simplify the settings on the automated nucleic acid preprocessing apparatus and reduce the user's expertise requirement, thereby reducing the labor costs and the user learning curve. In addition, the automated high-throughput preprocessing method and system of the present disclosure support multiple specifications of the sampling tubes, and also support multiple test settings for one single tube, so multiple tests can be performed on the same sample at the same time, and the information of the test plate configuration can also be output for use by the downstream PCR machine. Furthermore, in addition to the system's preset protocols, the user can also modify or customize new reagent formulas, test protocols, and extraction protocols according to the requirements, providing more flexible applications.

While the disclosure has been described in terms of what is presently considered to be the most practical and preferred embodiments, it is to be understood that the disclosure needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A high-throughput automated preprocessing method, applied to a nucleic acid preprocessing apparatus comprising a control system and three cabins connected in series and communicated with or separated from each other, wherein the three cabins serve as a sample transfer area, a nucleic acid extraction area, and a reagent setup area, respectively, and the control system comprises a user interface and guides a user to set up on the user interface, the high-throughput automated preprocessing method comprising steps of:
in the sample transfer area:
(a1) the user selecting a sampling tube type, a test protocol and an extraction protocol on the user interface;
(a2) the control system guiding the user to place consumables of a sampling tube, an extraction plate, and a tip;
(a3) the control system confirming configurations of the consumables, and performing a sample transfer task; and
(a4) after the sample transfer task is completed, the control system conveying the extraction plate to the nucleic acid extraction area from the sample transfer area,
in the nucleic acid extraction area:
(b1) after the extraction plate is conveyed to the nucleic acid extraction area, the control system performing a nucleic acid extraction task based on the selected extraction protocol; and
(b2) after the nucleic acid extraction task is completed, the control system conveying the extraction plate to the reagent setup area from the nucleic acid extraction area,
in the reagent setup area:
(c1) the control system guiding the user to place consumables of a test plate, a tip, a reagent plate and reagents;
(c2) the control system confirming configurations of the consumables, and performing a reagent deployment task based on the selected test protocol; and
(c3) after the extraction plate is conveyed to the reagent setup area, the control system performing a nucleic acid transfer task,
wherein the sample transfer area, the nucleic acid extraction area, and the reagent setup area are able to simultaneously perform preprocessing of different batches of samples.

2. The high-throughput automated preprocessing method according to claim 1, wherein the sampling tube type comprises information of a sample type, and the control system recommends the extraction protocol based on the sample type.

3. The high-throughput automated preprocessing method according to claim 1, wherein the control system automatically sets up a corresponding tube tray layout based on the selected sampling tube type.

4. The high-throughput automated preprocessing method according to claim 1, wherein the sample transfer task is to transfer the sample in the sampling tube to the extraction plate.

5. The high-throughput automated preprocessing method according to claim 1, wherein the control system confirms the configurations of the consumables through image recognition with a camera module to check whether sufficient and correct consumables are placed.

6. The high-throughput automated preprocessing method according to claim 1, in the sample transfer area, further comprising a step of scanning a tag on the sampling tube to confirm whether the sample is correct.

7. The high-throughput automated preprocessing method according to claim 1, wherein the nucleic acid transfer task is to transfer nucleic acids in the extraction plate to the test plate.

8. The high-throughput automated preprocessing method according to claim 1, in the reagent setup area, further comprising a step of sealing the test plate.

9. The high-throughput automated preprocessing method according to claim 1, wherein the control system provides a task monitoring function through the user interface, allowing the user to instantly monitor execution progresses in the sample transfer area, the nucleic acid extraction area, and the reagent setup area are.

10. The high-throughput automated preprocessing method according to claim 1, wherein the control system displays an abnormality notification on the user interface and guides the user to troubleshoot the abnormality in the respective area where the abnormality occurs.

11. A high-throughput automated preprocessing system, applied to a nucleic acid preprocessing apparatus comprising a control system and three cabins connected in series and communicated with or separated from each other, wherein the three cabins serve as a sample transfer area, a nucleic acid extraction area, and a reagent setup area, respectively, the control system comprises a user interface and guides a user to set up on the user interface, and the high-throughput automated preprocessing system is used to perform a high-throughput automated preprocessing method comprising steps of:
in the sample transfer area:
(a1) the user selecting a sampling tube type, a test protocol and an extraction protocol on the user interface;
(a2) the control system guiding the user to place consumables of a sampling tube, an extraction plate, and a tip;
(a3) the control system confirming configurations of the consumables, and performing a sample transfer task; and
(a4) after the sample transfer task is completed, the control system conveying the extraction plate to the nucleic acid extraction area from the sample transfer area,
in the nucleic acid extraction area:
(b1) after the extraction plate is conveyed to the nucleic acid extraction area, the control system performing a nucleic acid extraction task based on the selected extraction protocol; and
(b2) after the nucleic acid extraction task is completed, the control system conveying the extraction plate to the reagent setup area from the nucleic acid extraction area,
in the reagent setup area:
(c1) the control system guiding the user to place consumables of a test plate, a tip, a reagent plate and reagents;
(c2) the control system confirming configurations of the consumables, and performing a reagent deployment task based on the selected test protocol; and
(c3) after the extraction plate is conveyed to the reagent setup area, the control system performing a nucleic acid transfer task,
wherein the sample transfer area, the nucleic acid extraction area, and the reagent setup area are able to simultaneously perform preprocessing of different batches of samples.

12. The high-throughput automated preprocessing system according to claim 11, wherein the sampling tube type comprises information of a sample type, and the control system recommends the extraction protocol based on the sample type.

13. The high-throughput automated preprocessing system according to claim 11, wherein the control system automatically sets up a corresponding tube tray layout based on the selected sampling tube type.

14. The high-throughput automated preprocessing system according to claim 11, wherein the sample transfer task is to transfer the sample in the sampling tube to the extraction plate.

15. The high-throughput automated preprocessing system according to claim 11, wherein the control system confirms the configurations of the consumables through image recognition with a camera module to check whether sufficient and correct consumables are placed.

16. The high-throughput automated preprocessing system according to claim 11, in the sample transfer area, further comprising a step of scanning a tag on the sampling tube to confirm whether the sample is correct.

17. The high-throughput automated preprocessing system according to claim 11, wherein the nucleic acid transfer task is to transfer nucleic acids in the extraction plate to the test plate.

18. The high-throughput automated preprocessing system according to claim 11, in the reagent setup area, further comprising a step of sealing the test plate.

19. The high-throughput automated preprocessing system according to claim 11, wherein the control system provides a task monitoring function through the user interface, allowing the user to instantly monitor execution progresses in the sample transfer area, the nucleic acid extraction area, and the reagent setup area are.

20. The high-throughput automated preprocessing system according to claim 11, wherein the control system displays an abnormality notification on the user interface and guides the user to troubleshoot the abnormality in the respective area where the abnormality occurs.
